(19) United States
(12) Patent Application Publication
(10) Pub. No.: US 2022/0065847 A1
(43) Pub. Date: Mar. 3, 2022

(54) SAFE HANDLING LATER.AL FLOW IMMUNOASSAY CASSETTES FOR POINT OF CARE TESTING

(71) Applicant: Izhar Hasan, Monmouth Beach, NJ (US)

(72) Inventor: Izhar Hasan, Monmouth Beach, NJ (US)

(21) Appl. No.: 17/460,921

(22) Filed: Aug. 30, 2021

Related U.S. Application Data
(60) Provisional application No. 63/071,429, filed on Aug. 28, 2020.

(51) Int. Cl. G01N 33/53; B01L 3/00

(52) U.S. Cl. CPC G01N 33/5302; B01L 3/5027; B01L 2200/027; B01L 2200/0605; B01L 2200/082; B01L 2200/085; B01L 2300/0672; B01L 2300/069; B01L 2300/0609; B01L 2300/0825; B01L 2300/0864; B01L 2300/126; B01L 2400/0475; B01L 2300/025

(57) ABSTRACT

A point of care cassette, comprising: a housing; a lancet carrier for carrying a lancet, the lancet carrier being configured to be retractably urged by a spring from a locked position to a deployed position in response to an actuation of a first trigger, the lancet being entirely within the housing when not in the deployed position; and a test carrier for carrying a testing medium, the test carrier configured to be urged by a second spring from a locked position to a deployed position in response to an actuation of a second trigger, the second trigger being actuated during deployment of the lancet carrier to bring the testing medium proximate a blood sample after retraction of the lancet.

### CROSS-REFERENCE TO RELATED APPLICATION

This application claims priority to U.S. Provisional Patent Application No. 63/071,429 filed on Aug. 28, 2020 entitled SAFE HANDLING LATERAL FLOW IMMUNOASSAY CASSETTES FOR POINT OF CARE TESTING (Attorney Docket No. V0697), which application is incorporated herein by reference in its entirety.

### FIELD OF THE DISCLOSURE

The present disclosure generally relates to testing for medical and/or microbial conditions including viral, bacterial, and other related infectious diseases in an individual, and more particularly to safe handling lateral-flow-immunoassay (SHLFIA) cassettes that implement such testing.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Point of care testing (POCT) is medical testing implemented at or near the site of patient care. POCT results in rapid (and convenient) test results of a desired diagnostic suspicion to provide timely clinical care to the patient. Currently, POCT testing includes tests for various diseases such as metabolic/endocrine disease (blood glucose testing, thyroid stimulating hormone, blood gas and electrolytes analysis), blood diseases (e.g., rapid coagulation testing), acute heart conditions (e.g., cardiovascular rapid c markers diagnostics), drug treatment (e.g., drug screening), without limitation. POCTs are available for urine testing, pregnancy testing, fecal occult blood analysis, food pathogen screening, hemoglobin diagnostics, infectious disease testing, cholesterol screening, cancer testing (e.g. PSA), hormone testing (hCG, LH, FSH), cardiac state determinations (e.g., detecting troponin), pulmonary, gastroenterology (e.g., H. pylori antibodies), urology, dermatology, neurology, pediatrics, surgical, and public health (Ebola, cholera, HIV), testing and combinations thereof.

Bloodborne pathogens are any pathogenic microorganisms found in the blood or other bodily infectious material that can cause disease in humans. Examples of bloodborne pathogens include hepatitis B virus hepatitis C virus , human immunodeficiency virus (HIV), malaria, syphilis, viral hemorrhagic fever, arboviral infections, Creutzfeldt-Jakob disease, and relapsing fever. Most common bloodborne pathogens involved in occupational exposures in healthcare workers include hepatitis B, hepatitis C, and HIV A commonly employed conventional testing method for POCT involves the use of lateral flow assay (LFA) cassettes. Various biological samples can be tested using LFAs, including urine [Boisen M. L., Oottamasathien D., Jones A. B., Millett M. M., Nelson D. S., Bornholdt Z. A., et al. Development of prototype Filovirus recombinant antigen immunoassays. J. Infect. Dis. 2015;212(Suppl. 2):S359-367. doi: 10.1093/infdis/jiv353], saliva [Nielsen K., Yu W. L., Kelly L., Bermudez R., Renteria T., Dajer A., et al. Development of a lateral flow assay for rapid detection of bovine antibody to Anaplasma marginale. J. Immunoassay Immunochem. 2008;29:10-18. doi: 10.1080/15321810701734693], sweat [Rohrman B. A., Leautaud V., Molyneux E., Richards-Kortum R. R. A lateral flow assay for quantitative detection of amplified HIV-1 RNA. PLoS One. 2012;7:e45611. doi: 10.1371/journal.pone], serum [Kamphee H., Chaiprasert A., Prammananan T., Wiriyachaiporn N., Kanchanatavee A., Dharakul T. Rapid molecular detection of multidrug-resistant tuberculosis by PCR-nucleic acid lateral flow immunoassay. PLos One. 2015;10:e0137791. doi: 10.1371/journal.pone.0137791], plasma [Moreno M. L., Cebolla A., Munoz-Suano A., Carrillo-Carrion C., Comino I., Pizarro A., et al. Detection of gluten immunogenic peptides in the urine of patients with coeliac disease reveals transgressions in the gluten-free diet and incomplete mucosal healing. Gut. 2015 doi:10.1136/gutjnl-2015-310148], whole blood [Carrio A., Sampedro C., Sanchez-Lopez J. L., Pimienta M., Campoy P. Automated low-cost smartphone-based lateral flow saliva test reader for drugs-of-abuse detection. Sensors. 2015;15:29569-29593. doi: 10.3390/s151129569] and other body fluid types.

Various industries rely quite heavily on the use of LFAs to test various biological samples, such as veterinary medicine [Pacifici R., Farre M., Pichini S., Ortuno J., Roset P. N., Zuccaro P., et al. Sweat testing of MDMA with the Drugwipe analytical device: a controlled study with two volunteers. J. Anal. Toxicol. 2001;25:144-146. doi: 10.1093/jat/25.2.144], quality control [De Giovanni N., Fucci N. The current status of sweat testing for drugs of abuse: a review. Curr. Med. Chem. 2013;20:545-561] and environmental health and safety [Magambo K. A., Kalluvya S. E., Kapoor S. W., Seni J., Chofle A. A., Fitzgerald D. W., et al. Utility of urine and serum lateral flow assays to determine the prevalence and predictors of cryptococcal antigenemia in HIV-positive outpatients beginning antiretroviral therapy in Mwanza, Tanzania. J. Int. AIDS Soc. 2014;17:19040. doi: 10.7448/IAS.17.1.19040]. Conventional rapid LFA cassette tests are utilized to screen for animal diseases [Schramm E. C., Staten N. R., Zhang Z., Bruce S. S., Kellner C., Atkinson J. P., et al. A quantitative lateral flow assay to detect complement activation in blood. Anal. Biochem. 2015;477:78-85. doi: 10.1016/j.ab.2015.01.024], pathogens [Ang S. H., Rambeli M., Thevarajah T. M., Alias Y. B., Khor S. M. Quantitative, single-step dual measurement of hemoglobin A1c and total hemoglobin in human whole blood using a gold sandwich immunochromatographic assay for personalized medicine. Biosens. Bioelectron. 2015;78:187-193. doi: 10.1016/j.bios.2015.11.045], chemicals [Nielsen K., Yu W. L., Kelly L., Williams J., Dajer A., Gutierrez E., et al. Validation and field assessment of a rapid lateral flow assay for detection of bovine antibody to Anaplasma marginale. J. Immunoassay Immunochem. 2009;30:313-321. doi: 10.1080/15321810903084749], toxins [van Dam G. J., de Dood C. J., Lewis M., Deelder A. M., van Lieshout L., Tanke H. J., et al. A robust dry reagent lateral flow assay for diagnosis of active schistosomiasis by detection of Schistosoma circulating anodic antigen. Exp. Parasitol. 2013;135:274-282. doi: 10.1016/j.exppara.2013.06.017] and water pollutants [Ching K. H., He X., Stanker L. H., Lin A. V., McGarvey J. A., Hnasko R. Detection of shiga toxins by lateral flow assay. Toxins. 2015;7:1163-1173. doi: 10.3390/toxins7041163].

Please also see www.ceufast.com/course/osha-occupational-exposure-to-blood-borne-pathogen, Rhode, K. A., Dupler, A. E., Postma, J., Sanders A. (2013).Minimizing nurses' risks for needlestick injuries in the hospital setting. Workplace Health & Safety. 61 (5), 197-202; Karmon, S. L., Mehta, S. A., Brehm, A., Dzurenko, J., Phillips, M. (2013). Evaluation of bloodborne pathogen exposures at an urban hospital. American Journal of Infection Control. 41(2), 185-186; Henderson, D. K. (2012). Management of needlestick injuries. A house officer who has a needlestick. Journal of the American Medical Association. 37 (1), 75-84; and Bernard, J. A., Datillo, J. R., LaPorte, D. M. (2013). The incidence and reporting of sharps exposure among medical students, orthopedic residents, and faculty at one institution. Journal of Surgical Education. 70 (5), 660-668.

Typically, known lateral flow assay (LFA) cassettes that implement POCT testing are simple plastic cases with a test strip, such as depicted in prior art FIG. 1. The known LFA test strip includes a sample pad 10 for receiving fluid (e.g., droplets 12), a conjugate release pad 14, a membrane 16 with immobilized antibodies, test line 18 and control line 20 and an adsorbent pad 22. The components of the LFA strip are usually fixed to an inert backing material 24.

Techniques associated with conventional LFA testing are sometimes referred to as lateral flow chromatographic immunoassay methods or techniques. Such techniques (and related know devices) are described in detail in U.S. Pat. Nos. 4,956,302, 6,764,825, 5,008,080, 6,183,972 and WIPO Pub. No WO 90/06511, each of which are incorporated herein by reference.

The known underlying biochemical mechanism in these known LFAs facilitates reactions between antibodies and antigens, commonly to as a specific binding assay. Other examples of underlying biochemical mechanisms in known LFAs include binding reaction of DNA and RNA hybridization, which typically involve hormones and other biological receptors.

A typical whole blood POCT sample collection requires a lancet to pierce a finger of the person under test, followed by either manual squeezing of the finger or using a fluid collecting pipette to capture the whole blood. Then, the captured whole blood is transferred to a sample pad of the test strip. FIG. 2 depicts several known procedures for POCT blood sample testing. The first procedure may be referred to as a whole blood/hanging drop procedure. The second procedure may be referred to as a whole blood capillary tube procedure. The third procedure is referred to as a venipuncture, serum or plasma procedure. Often, a retractable lancet is used during these procedures for safety reasons to avoid common finger pricking injury for blood borne pathogen exposure risk in health care professionals. The retractable lancet is a spring-loaded needle which retracts into the body of the device after skin puncture. As is known, all lancets are sterile and for one-time use only.

### SUMMARY

Various deficiencies in the prior art are addressed by apparatus such as safe handling lateral-flow immunoassay (SHLFIA) cassettes that implement point of care testing (POCT) suitable for implementing point of care testing (POCT). Exemplary SHLFIA embodiments provide improved safety while saving time as compared to existing point of care diagnostic (POCT) cassettes by, for example, enabling in a single procedural event the hazardous and time consuming steps of blood collection (in reliance upon a lancet) and the transfer of collected blood to a test strip in the cassette. That is, the inventive SHLFIA cassettes implement the conventional steps of capturing a blood sample, and then transferring the captured blood sample to a test strip in one step not only for convenience and expediency, but also for safety. Handling of the fluid (e.g., blood sample) is minimized.

In various embodiments, the inventive safe handling lateral-flow immunoassay (SHLFIA) cassettes implement point of care testing in reliance upon a test strip modified to include at least one built in capillary tube for blood volume transfer, according to the inventive principles.

The various disclosed SHLFIAs provide a rapid access at point-of-care/need. Advantageously, the various disclosed SHLFIA cassettes are inexpensive to manufacture as well as easy to use, making them desirable in many industries that rely on point of care testing.

The various disclosed SHLFIAs are well-suited for screening a medical professional's initial diagnostic suspicion at point of care of a patient in ambulatory or home settings for quick “next” clinical care management decisions.

Various embodiments include a point of care cassette, comprising: a housing; a lancet carrier for carrying a lancet, the lancet carrier being configured to be retractably urged by a spring from a locked position to a deployed position in response to an actuation of a first trigger, the lancet being entirely within the housing when not in the deployed position; and a test carrier for carrying a testing medium, the test carrier configured to be urged by a second spring from a locked position to a deployed position in response to an actuation of a second trigger, the second trigger being actuated during deployment of the lancet carrier to bring the testing medium proximate a blood sample after retraction of the lancet.

Additional objects, advantages, and novel features of the invention will be set forth in part in the description which follows, and will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 depicts a prior art lateral flow assay (LFA) cassette;

FIG. 2 depicts prior art blood sample collection methodologies for collecting and transferring blood to a conventional point of care diagnostic cassette;

FIG. 3 depicts a top perspective view of a safe handling lateral-flow immunoassay (SHLFIA) cassette for implement point of care testing (POCT) in accordance with the inventive principles;

FIG. 4 depicts a bottom perspective view of a safe handling lateral-flow immunoassay (SHLFIA) cassette depicted in FIG. 3;

FIG. 5 depicts an inside view of the safe handling lateral-flow immunoassay (SHLFIA) cassette depicted in FIG. 3, where the bottom half of the housing is removed for clarity;

FIG. 6 depicts the lancet assembly in the safe handling lateral-flow immunoassay (SHLFIA) cassette depicted in FIG. 3, in a close-up detailed view;

FIG. 7 depicts a side view of the safe handling lateral-flow immunoassay (SHLFIA) cassette depicted in FIG. 5, to highlight a second trigger;

FIG. 8 depicts a side view of the safe handling lateral-flow immunoassay (SHLFIA) cassette depicted in FIG. 5, to highlight operation when the spring is compressed;

FIG. 9 depicts a side view of the safe handling lateral-flow immunoassay (SHLFIA) cassette depicted in FIG. 5, to highlight operation where the second trigger and compressed spring moves a test strip carrier including a test strip forward; and

FIG. 10 depicts a side view of the safe handling lateral-flow immunoassay (SHLFIA) cassette depicted in FIG. 5, to highlight operation where the test strip is exposed past the end and takes the fluid sample.

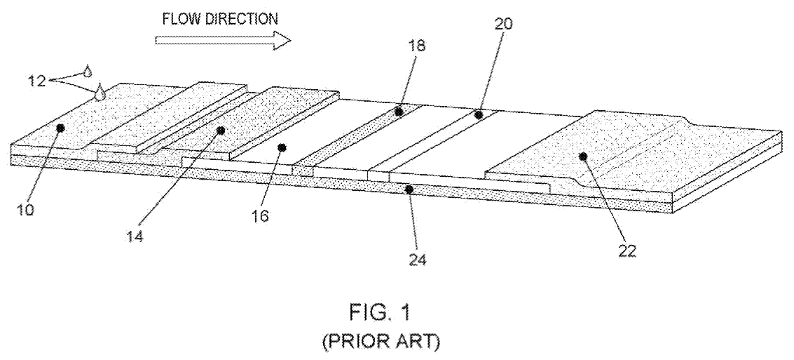

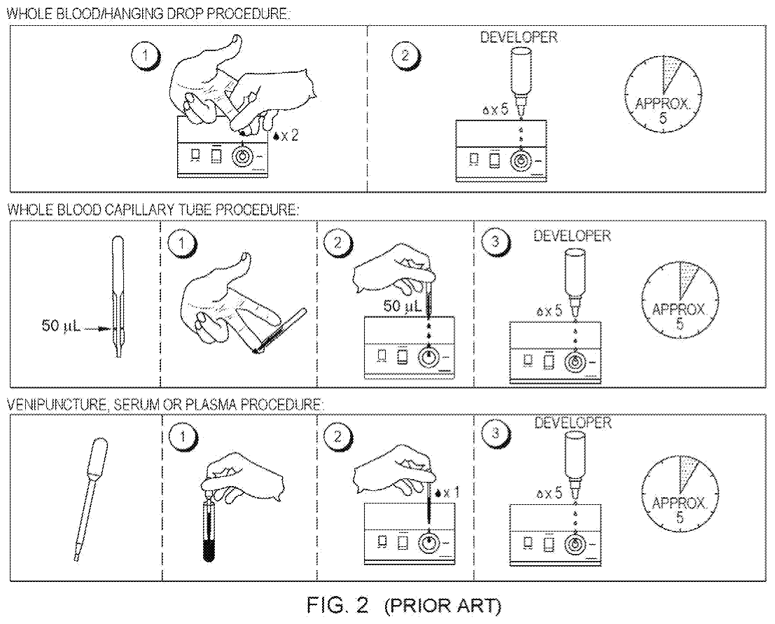

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for pedagogical purposes to aid the reader in understanding the principles of the invention and the concepts contributed by the inventor(s) to furthering the art, and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Given the growing concern for safety in bodily fluid testing, it is seen to be desirable to obtain a safe and expedient result at point of care testing that avoids the multiple steps required to implement conventional fluid capture and testing.

Various deficiencies in the prior art are addressed by apparatus such as safe handling lateral-flow immunoassay (SHLFIA) cassettes that implement point of care testing (POCT) suitable for implementing point of care testing (POCT). Exemplary SHLFIA embodiments provide improved safety while saving time as compared to existing point of care diagnostic (POCT) cassettes by, for example, enabling in a single procedural event the hazardous and time consuming steps of blood collection (in reliance upon a lancet) and the transfer of collected blood to a test strip in the cassette. That is, the inventive SHLFIA cassettes implement the conventional steps of capturing a blood sample, and then transferring the captured blood sample to a test strip in one step not only for convenience and expediency, but also for safety. Handling of the fluid (e.g., blood sample) is minimized.

In various embodiments, the inventive safe handling lateral-flow immunoassay (SHLFIA) cassettes implement point of care testing in reliance upon a test strip modified to include at least one built in capillary tube for blood volume transfer, according to the inventive principles.

The various disclosed SHLFIAs provide a rapid access at point-of-care/need. Advantageously, the various disclosed SHLFIA cassettes are inexpensive to manufacture as well as easy to use, making them desirable in many industries that rely on point of care testing.

The various disclosed SHLFIAs are well-suited for screening a medical professional's initial diagnostic suspicion at point of care of a patient in ambulatory or home settings for quick “next” clinical care management decisions.

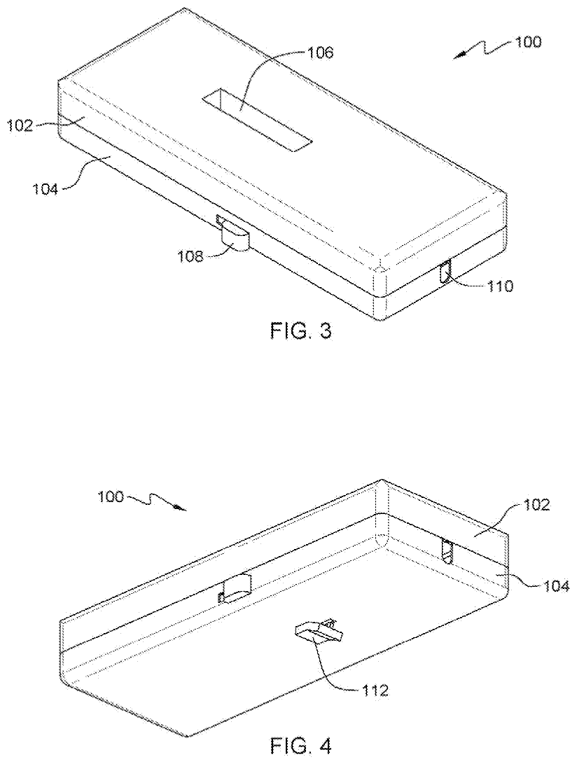

FIGS. 3-10 depict various views of exemplary embodiment of the inventive safe handling lateral-flow immunoassay (SHLFIA) cassette and, as such, will be described together.

FIG. 3 depicts an exemplary embodiment of the inventive safe handling lateral-flow immunoassay (SHLFIA) cassette, for implementing point of care testing (POCT) in accordance with the inventive principles. As depicted in FIG. 3, the SHLFIA includes a housing comprising a top housing part 102 and a bottom housing part 104. The top housing part 102 includes an opening 106 for reading a test strip 114 contained therein. The bottom housing part 104 of the housing includes an opening to enable a load button assembly 108 having components passing therethrough to be slidably engaged with the bottom housing part 104 for “loading” the lancet assembly 118/lancet 119. The bottom housing part 104 of the housing further includes an opening 110 through which a lancet 119 (not shown in FIG. 3) is subsequently deployed to pierce the skin and capture blood; the lancet is retracted after its job is done. The loading prepares the lancet by moving it against the urging of a spring 120 and into a “cocked” position, wherein the lancet holds the spring 120 in a compressed state under the control of a trigger 112. Upon a triggering event (i.e., trigger actuation), the trigger 112 mechanism is moved such that it stops inhibiting motion of the lancet and thereby enables the compressed spring 120 to expand and move the lancet toward a “deployed” position for penetrating the skin of a patient.

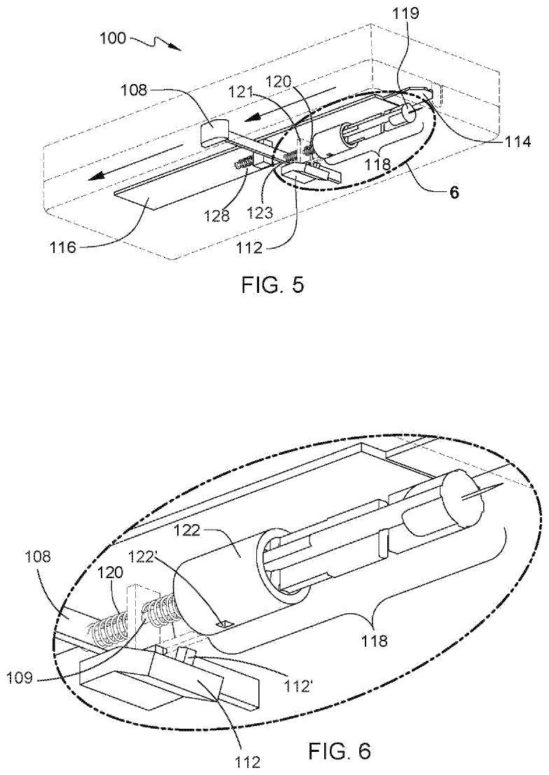

FIG. 4 depicts a bottom perspective view of the safe handling lateral-flow immunoassay (LFIA) cassette 100 depicted in FIG. 3, to highlight operation of trigger 112 arranged in the bottom housing part 104 of the housing. In operation, load button assembly 108 is pulled back to “cock” or ready the LFIA cassette for its intended operation; namely, the lancet needle is arranged in a lading, cocked, or loaded position to prepare for the triggering event (i.e., actuation of trigger 112). When trigger 112 is pushed or otherwise actuated, the lancet (see FIGS. 5-6) moves forward, extends out of the opening 110 and punctures the skin of the fingertip of the person under test. The lancet is returned to its non-deployed position within the housing and the test strip is compelled forward to collect the blood (fluid) sample.

FIG. 5 depicts an inside view of the safe handling lateral-flow immunoassay (SHLFIA) cassette depicted in FIGS. 3 and 4; the bottom half of the housing is removed for clarity. As shown therein, test strip 114 is loaded into a test strip carrier 116. The load button assembly 108 is slid back and in turn pushes back test strip carrier 116/test strip 114 (thereby compressing spring 128) and a lancet assembly 118 (thereby compressing spring 120), the lancet assembly 118 including a lancet carrier 122 comprising a piston-like element having an opening configured to accept insertion thereinto of a lancet 119 having a shape compatible with the lancet carrier opening (e.g., a cylindrical base disposable lancet inserted into a cylindrical opening of a lancet carrier). There is a small opening in the bottom housing part 104 that allows a “pin” 112′ on trigger 112 to pass through and lock into a recess 122′ in the lancet carrier 122 of the lancet assembly 118 so as to hold the lancet assembly 118 in the cocked position against the urging of spring 120. It can be seen by inspection that the

FIG. 6 depicts the lancet assembly 118 in the safe handling lateral-flow immunoassay (SHLFIA) cassette 100 depicted in FIG. 5, in a close-up detailed view. The lancet assembly 118 includes the lancet carrier 122 mechanically cooperating with the spring 120 such that when trigger when triggered by trigger 112 (i.e., when the trigger 112 pivots such that the trigger pin 112′ is removed from the recess 122′ of the lancet carrier 122), the spring 120 releases its stored spring energy to compel or urge the lancet assembly 118 forward toward the opening in the housing.

It can be seen by inspection of FIGS. 5-6 that the loading assembly 108 is coupled to the lancet carrier 122 of the lancet assembly 118 via a connecting rod 109, which further serves as a guide rod for springs 120 and 123 by axially passing therethrough, the springs 120/123 shown as being separated by a spring separator 121.

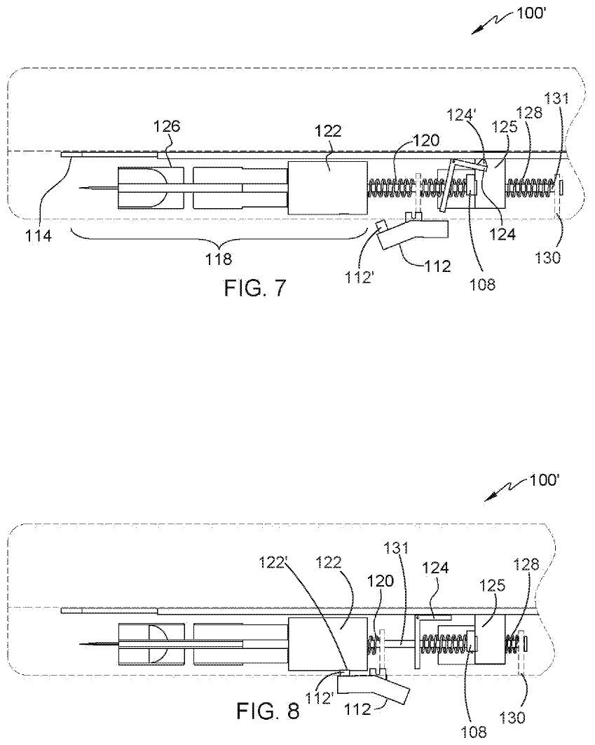

FIG. 7 depicts a side view of the safe handling lateral-flow immunoassay (SHLFIA) cassette 100′, which is distinguishable from the SHLFIA cassette 100 depicted in FIG. 5, as it includes a second (internal) trigger 124. As such, when SHLFIA cassette 100′ is cocked (i.e., the lancet assembly is cocked and the protrusion of the first trigger 112 prevents deployment of the lancet assembly 118 by the spring 120), the second trigger 124 is configured to lock into a recess in the bottom of the test strip carrier 116.

As shown n FIG. 7, the lancet assembly 118 is in the loaded or cocked position (note the protrusion of, and position of, first trigger 112 inhibiting further motion of the spring 120). The second trigger 124 is depicted in an unengaged position (i.e., not being locked into the recess in the bottom of test strip carrier 116).

It can be seen by inspection that the spring 120 has a proximal portion mechanically cooperating with the lancet carrier 122, and a distal portion mechanically cooperating with a front-facing surface of the load button assembly 108. Further, a rear facing surface of the load button assembly 108 is mechanically cooperating with a second piston-like element 125 so as to compress a second spring 128 disposed between the second piston-like element 125 and an anchoring point 130.

It can be seen by inspection that the second trigger 124 is pivotally engaged with the first spring 120 at a location near the distal portion of the spring 120. As the first spring 120 is compressed during the cocking of the lancet assembly 118, the first spring 120 imparts a force upon the second trigger 124 which urges the protruding member 124′ into a corresponding recess in the underside of the test strip carrier 116 to lock the test strip carrier to the second switch 124, as depicted in FIG. 8. When not aligned with the recess under the test strip carrier 116 (as depicted in FIG. 7), the point of the protruding member merely contacts and slides along the underside of the test strip carrier 116 as the spring 120 is compressed.

It can be seen by inspection that a spring guide or rod 131 is secured at anchoring point 130 and extends axially through the second spring 128 and is slidably engaged with the second piston-like member 125.

FIG. 8 depicts a side view of the safe handling lateral-flow immunoassay (SHLFIA) cassette 100 depicted in FIG. 5, to highlight inventive operation when the spring 120 is compressed. Pressing trigger 112 releases the lancet assembly 118, where the spring 120 moves it forward, i.e., deploys the lancet assembly to break the skin and capture a blood sample.

It is noted that the blood sample captured by the lancet may be delivered to the testing medium 114 directly, or that the testing medium 114 may be brought into contact with blood emerging at the puncture site of the skin pierced by the lancet 119. The testing medium 114 may comprise a test strip sample pad, a microfluid based biological chip, or other testing medium. Fluid transfer to the testing medium may be via a fluid channel of microfluid based biological chip, a capillary tube configured to receive a blood sample thereby and/or a plurality of capillary tubes. Further, the one or more fluid channels of a microfluid based biological chip, or capillary tubes for other media may be sized to collect a predetermined amount of blood, such as for a test for a specific disease or condition.

It can be seen by inspection that the protruding member 124′ of the second switch 124 has now moved into a position whereby the protruding member 124′ is now aligned and inserted into the recess under the test strip carrier 116. It can also be seen by inspection that the second spring 128 has now been compressed between the second piston-like element 125 and the anchoring point 130. That is, operation of the load button assembly 108 being moved backward has resulted in compression of spring 120 until the first trigger is locked (thereby cocking the lancet assembly 118) and spring 128 until the second trigger is locked (thereby cocking the test carrier 116/test strip 114 assembly).

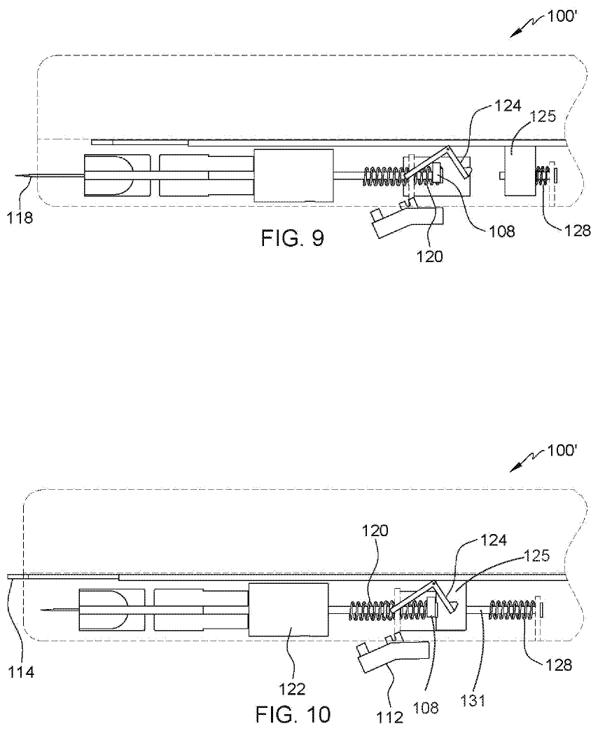

FIG. 9 depicts a side view of the safe handling lateral-flow immunoassay (SHLFIA) cassette 100′ depicted in FIG. 7, to highlight operation where the second trigger 124 and compressed spring 120 moves the test strip carrier 116/test strip 114 forward. The FIG. 9 depicts the distal portion of the spring 120 as compressed. The compression stores spring energy that operates to deploy and withdraw the lancet assembly 118 when the trigger 112 is actuated.

Lancet extension/retraction. After the load button assembly 108 has compressed springs 120 and 128 to cock the lancet assembly 118 and test carrier 116/test strip 114 assembly (FIG. 8). Actuating trigger 112 releases the cocked lancet assembly 118, whereupon the compressed proximate end of the spring 120 urges the lancet assembly 118 forward such that the lancet 119 protrudes beyond the housing and punctured the skin of the patient. It is noted that the spring 120 operates to extend the lancet 119 beyond the housing, as which point the spring 120 is in an over-extended state such that the spring 120 acts to withdraw the lancet 119 back into the housing.

FIG. 10 depicts a side view of the lateral-flow immunoassay (LFIA) cassette 100′ depicted in FIGS. 7, 8 and 9, to highlight operation where the test strip 114 is exposed past the end of the housing so as to contact and take the fluid sample.

Test carrier 116/test strip 114 extension. During the action of the lancet being extended, the load button assembly 108 is carried forward such that it urges the second switch 124 forward, which causes the second switch 124 to pivot such that the protrusion 124′ disengages from the underside of carrier 116. This enables the second piston-like element 125 to move forward along with the test carrier 116/test strip 114 assembly such that the test strip 114 protrudes from the housing to contact the skin of the patient proximate to where the lancet 119 had penetrated the skin to draw blood to be sampled.

Thus, various embodiments provide a point of care cassette, comprising a housing; a lancet carrier for carrying a lancet, the lancet carrier being configured to be retractably urged by a spring from a locked position to a deployed position in response to an actuation of a first trigger, the lancet being entirely within the housing when not in the deployed position; and a test carrier for carrying a testing medium, the test carrier configured to be urged by a second spring from a locked position to a deployed position in response to an actuation of a second trigger, the second trigger being actuated during deployment of the lancet carrier to bring the testing medium proximate a blood sample after retraction of the lancet. The testing medium may comprise a test strip sample pad, a microfluid based biological chip, or other testing medium. The medium may be made any kind of test medium, such as those made of paper or other absorbent materials. Fluid transfer to the testing medium may be via a fluid channel of microfluid based biological chip, a capillary tube configured to receive a blood sample thereby and/or a plurality of capillary tubes (especially useful for non-absorbent testing media). Further, the one or more fluid channels of a microfluid based biological chip, or capillary tubes for other media may be sized to collect a predetermined amount of blood, such as for a test for a specific disease or condition.

Various embodiments described above include a point of care cassette, comprising a retractable lancet; a capillary tube in fluid communication with the lancet; a test strip sample pad comprising diagnostic testing strips; and a trigger; wherein upon actuating the trigger, the retractable lancet and capillary tube operate together to capture and transport a blood sample from a fingertip to the test strip sample pad in a single operation. In some embodiments of this point of care cassette, the lancet is deployed to pierce skin at the fingertip, capture the blood sample in the capillary tube, retract the lancet and eject the captured blood sample from the capillary tube to the test strip sample pad or a fluid channel of microfluid based biological chip. In some embodiments of this point of care cassette, the diagnostic testing strips comprising the test strip sample pad are paper based. In some embodiments of this point of care cassette, the diagnostic testing strips are replaced by a microfluid based biochip for diagnostic assay of a desired disease assay. In some embodiments of this point of care cassette, the test strip of the test strip sample pad includes built-in capillary tubes of various sizes for customized blood volume transfer associated with various disease types.

As will be evident to persons skilled in the art, the foregoing detailed description, applications and figures are presented as examples of the invention, and that variations are contemplated that do not depart from the fair scope of the teachings and descriptions set forth in this disclosure.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like. It will be appreciated that the term “or” as used herein refers to a non-exclusive “or,” unless otherwise indicated (e.g., use of “or else” or “or in the alternative”).

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof.

What is claimed is:

1. A point of care cassette, comprising:
a housing;
a lancet carrier for carrying a lancet, the lancet carrier bring configured to be retractably urged by a spring from a locked position to a deployed position in response to an actuation of a first trigger, the lancet being entirely within the housing when not in the deployed position; and
a test carrier for carrying a testing medium, the test carrier configured to be urged by a second spring from a locked position to a deployed position in response to an actuation of a second trigger, the second trigger being actuated during deployment of the lancet carrier to bring the testing medium proximate a blood sample after retraction of the lancet.

2. The point of care cassette of claim 1, wherein the testing medium comprises a test strip sample pad.

3. The point of care cassette of claim 1, wherein the testing medium comprises a fluid channel of microfluid based biological chip.

4. The point of care cassette of claim 1, wherein the test strip comprises as least one capillary tube configured to receive a blood sample thereby.

5. The point of care cassette of claim 1, wherein the capillary tube is sized to collect a predetermined amount of blood.

6. The point of care cassette of claim 1, wherein the test strip comprises a plurality of capillary tubes sized to collect a predetermined amount of blood.

7. The point of care cassette of claim 1, wherein the test strip sample pad and at least one diagnostic testing strip disposed thereon are paper based.

8. The point of care cassette of claim 3, wherein the microfluid based biological chip comprise a diagnostic assay of a desired disease.

9. The point of care cassette of claim 2, wherein a test strip of the test strip sample pad includes built-in capillary tubes of various sizes for customized blood volume transfer associated with various disease types.

10. The point of care cassette of claim 1, wherein a test strip of the test strip sample pad includes built-in capillary tubes sized to transfer an amount of blood volume associated with an assay for a disease of interest.

11. Apparatus for obtaining a blood sample, comprising:
a housing;
a lancet carrier for carrying a lancet, the lancet carrier being configured to be retractably urged by a spring from a locked position to a deployed position in response to an actuation of a first trigger, the lancet being entirely within the housing when not in the deployed position; and
a test carrier for carrying a testing medium, the test carrier configured to be urged by a second spring from a locked position to a deployed position in response to an actuation of a second trigger, the second trigger being actuated during deployment of the lancet carrier to bring the testing medium proximate a blood sample after retraction of the lancet.
